(19) United States
(12) Patent Application Publication
(10) Pub. No.: US 2024/0058474 A1
(43) Pub. Date: Feb. 22, 2024

(54) CANNABINOID EMULSIONS AND COMPLEXES AND RELATED METHODS OF MANUFACTURE

(71) Applicant: E. & J. Gallo Winery, Modesto, CA (US)

(72) Inventor: Mark A. Kelm, Fresno, CA (US)

(21) Appl. No.: 18/451,402

(22) Filed: Aug. 17, 2023

Related U.S. Application Data
(60) Provisional application No. 63/398,693, filed on Aug. 17, 2022.

(51) Int. Cl. A61K 47/69; A61K 31/00; A61K 9/107; A61K 47/14

(52) U.S. Cl. CPC A61K 47/6951; A61K 31/658; A61K 9/107; A61K 47/14

(57) ABSTRACT

In general, the subject matter described herein relates to dry cannabinoid emulsions (DCEs), dried cannabinoid complexes, and related systems and methods. An example method of forming a DCE includes: obtaining a water phase; obtaining an oil phase including a cannabinoid; forming an oil-in-water emulsion in which the oil phase is suspended as particles in the water phase; and drying the emulsion in a refractance window dryer. An example method of forming a dried cannabinoid-cyclodextrin complex includes: obtaining an aqueous solution including cyclodextrin; adding a mixture of ethanol and a cannabinoid to the aqueous solution to form a cannabinoid-cyclodextrin complex; and drying the cannabinoid-cyclodextrin complex in a refractance window dryer.

### CROSS-REFERENCE TO RELATED APPLICATIONS

This application claims the benefit of U.S. Provisional Patent Application No. 63/398,693, filed Aug. 17, 2022, the entire contents of which are incorporated by reference herein.

### TECHNICAL FIELD

The present disclosure relates in general to the field of cannabinoid products and, in particular, to systems and methods for producing products containing cannabinoid emulsions and/or cannabinoid complexes.

### BACKGROUND

Oil-in-water (O/W) emulsions include a fine dispersion of minute droplets of oil evenly dispersed in water or other aqueous matrix. The oil may function as a carrier for hydrophobic substances, which may include active ingredients (“actives”). Emulsifiers, surfactants, stabilizers, carriers, and other components can be added to facilitate or improve stability of the emulsion and/or alter rheological properties.

Cannabinoids may include any of 100+ natural cannabinoids (e.g., cannabidiol (CBD), cannabinol (CBN), cannabigerol (CBG), and tetrahydrocannabinol (THC) isomers), endocannabinoids or analogs (e.g., anandamide), synthetic cannabinoids (e.g., HU-210), and mixtures thereof. Cannabinoids have been used to treat chronic pain, nausea due to chemotherapy, multiple sclerosis (MS)-related spasticity, and other medical conditions.

### SUMMARY

Certain processes used to produce cannabinoid emulsions and cannabinoid-cyclodextrin complexes are known; however, such emulsions and complexes are typically spray dried, which can result in a dull, non-flowable, fluffy, fine powder-like material with a high degree of surface area. As described herein, refractance window drying (RWD) technology can provide a glassy solid or “crystal” of emulsified cannabinoid and/or a dried cannabinoid-cyclodextrin complex. Such products made using RWD can be more flowable and more stable. The products can also be more dispersible into aqueous media, compared to powders which tend to clump and take time to fully disperse.

In various examples, the systems and methods described herein can produce a dry cannabinoid emulsion (DCE) (e.g., an oil-in-water emulsion that is dry to the touch) using RWD technology. The DCEs can be more physically and chemically stable than typical liquid emulsions. Such cannabinoid products can be more flowable and dispersible, and can possess a shiny luster relative to other drying techniques (e.g., spray drying). Products prepared using RWD can be less sensitive to degradation, for example, due to decreased surface area and/or larger particle sizes (e.g., a lower surface area to volume ratio), relative to spray-dried materials.

In some implementations, the systems and methods described herein can produce dried cannabinoid-gamma cyclodextrin (DC-GCD) complexes using RWD. The gamma-cyclodextrin can encapsulate CBD or other actives, and use of RWD can minimize loss of actives during the drying process. Alternatively, other cyclodextrins (e.g., delta cyclodextrins) may be used to encapsulate the actives.

In one aspect, the subject matter of this disclosure relates to a method of forming a dry cannabinoid emulsion (DCE). The method includes: obtaining a water phase; obtaining an oil phase including a cannabinoid; forming an oil-in-water emulsion in which the oil phase is suspended as particles in the water phase; and drying the emulsion in a refractance window dryer to form a dry cannabinoid emulsion (DCE).

In another aspect, the subject matter of this disclosure relates to a dry cannabinoid emulsion (DCE) composition. The composition includes: a matrix phase including a bulking agent and water; and particles of an oil phase including a cannabinoid and dispersed within the matrix phase. The particles can range in size from about 10 nm to about 400 nm.

In another aspect, the subject matter of this disclosure relates to a method of forming a dried cannabinoid-cyclodextrin complex. The method includes: forming an aqueous solution including cyclodextrin; adding a mixture of ethanol and a cannabinoid to the aqueous solution to form a cannabinoid-cyclodextrin complex; and drying the cannabinoid-cyclodextrin complex in a refractance window dryer to form a dried cannabinoid-cyclodextrin complex.

In another aspect, the subject matter of this disclosure relates to a composition. The composition includes: a dried cannabinoid-cyclodextrin complex; and at least one of an amylase enzyme, an excipient, or a bulking agent mixed with the dried cannabinoid-cyclodextrin complex.

These and other objects, along with advantages and features of embodiments of the present invention herein disclosed, will become more apparent through reference to the following description, the figures, and the claims. Furthermore, it is to be understood that the features of the various embodiments described herein are not mutually exclusive and can exist in various combinations and permutations.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings.

FIG. 1 is a schematic diagram of a method of producing a DCE, in accordance with embodiments described herein.

FIG. 2 is a schematic diagram of a method of producing a cannabinoid complex formulation, in accordance with embodiments described herein.

### DETAILED DESCRIPTION

It is contemplated that apparatus, systems, methods, and processes of the claimed invention encompass variations and adaptations developed using information from the embodiments described herein. Adaptation and/or modification of the apparatus, systems, methods, and processes described herein may be performed by those of ordinary skill in the relevant art.

It should be understood that the order of steps or order for performing certain actions is immaterial so long as the invention remains operable. Moreover, two or more steps or actions may be conducted simultaneously.

In various examples, the subject matter described herein relates to dry cannabinoid emulsions (DCEs), cannabinoid complexes (e.g., DC-GCD complexes), and systems and methods for producing the DCEs and cannabinoid complexes.

As used herein, a “dry cannabinoid emulsion” or “DCE” can be or include a composition that includes particles of an oil phase containing a cannabinoid (e.g., CBD) dispersed within a matrix phase that includes water and a filler or bulking agent (e.g., maltodextrin), which can act as a carrier. The DCE material itself can be in the form of particles, for example, with each DCE particle containing the oil phase and the matrix phase.

As used herein, a “cannabinoid complex” can be or include a molecular structure in which a cannabinoid molecule (e.g., a CBD molecule) is encapsulated or entrapped within another molecule (e.g., cyclodextrin). For example, a cyclodextrin molecule can have a hydrophilic surface and a hydrophobic cavity in which a cannabinoid molecule can be entrapped, to form a “cannabinoid-cyclodextrin complex.”

### Dry Cannabinoid Emulsions (DCE)

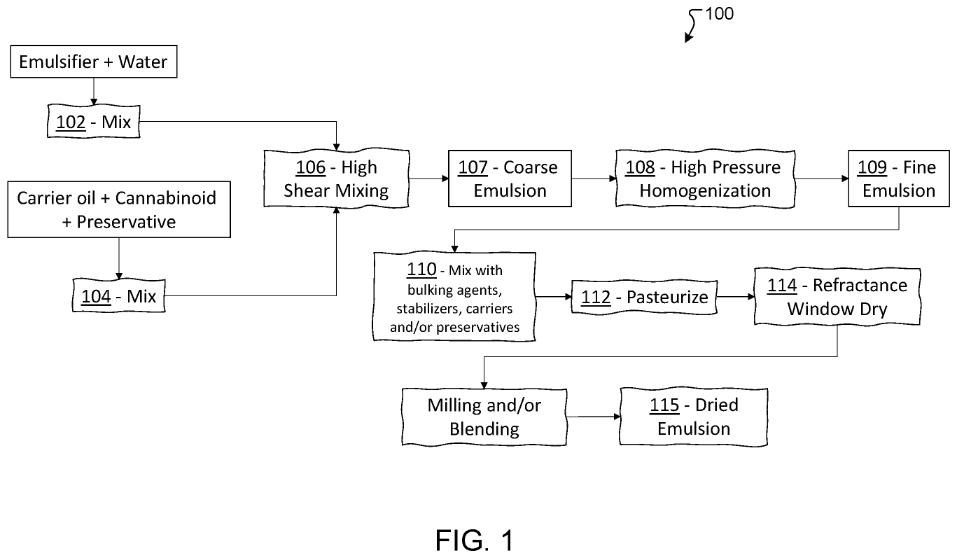

FIG. 1 includes a schematic diagram of an example method 100 of producing a DCE. In general, the method 100 involves producing a coarse O/W emulsion in a high-speed mixer, producing a fine O/W emulsion in a high-pressure homogenization device, and drying the fine O/W emulsion in an RWD process to produce the DCE.

At step 102, a water phase is produced by mixing an emulsifier with water. The emulsifier can be dissolved in the water by applying heat (e.g., heating to about 50° C.), waiting for a period of time (e.g., about 2 hours), and/or cooling the mixture. The emulsifier can be or include, for example, a biosurfactant (e.g., surface active substances from bacteria, yeast, or fungi, such as rhamnolipids or sophorolipids), a sucrose ester (e.g., sucrose laurate), a phospholipid (e.g., soy, canola, and/or sunflower lecithin), a protein (e.g., gelatin, pea protein, or potato protein), a polysaccharide (e.g., potato starch or gums), and/or a saponin (e.g., yucca saponin, yarn saponin, ginseng saponin, legume saponin, tea saponin, glycyrrhizin saponin, licorice root saponin, red beet saponin, oat bran saponin, or a quillaja saponin, such as O-NATURALE, available from INGREDION, or quillaja extract powder, available from GURADA). Similar to sucrose esters, O-alkylated organic or fatty acid esters may be derived from synthetic and/or natural sources to produce an effective emulsifier (e.g., propylene glycol fatty acid esters, ethoxylated jojoba esters, or esters of citric, tartaric, ascorbic, and other organic acids). Amide analogues of esters, proteins, and peptides can also function as emulsifiers. Sucrose esters can be colorless, odorless, tasteless, and non-allergenic, and these desirable properties can be conferred to the DCE and/or a final product produced using the DCE. Use of a saponin (a surface-active compound) or quillaja extract as the emulsifier can be advantageous for promoting emulsion stability, particularly when the emulsion is mixed with acidic substances (e.g., an acidic beverage base). A saponin or quillaja extract emulsifier can satisfy a desired to use natural ingredients, and can achieve a higher loading of actives in the emulsion (e.g., greater than 10%, on a dry basis). Compared to other emulsifiers, saponins (e.g., quillaja extract) are generally non-allergenic and/or less likely to cause an allergic reaction for consumers of the DCE or related products. The emulsifier and water can be combined at a ratio of about 1:150 (e.g., about 0.7% emulsifier), by weight, though other proportions can be used (e.g., from about 1:15 to about 1:1500). In various examples, the emulsifier can be or include one or more surface active substances that facilitate formation of emulsions by lowering an oil-water interfacial tension and/or imparting short-term stability by forming a protective film around emulsion droplets.

At step 104, an oil phase is produced by mixing a carrier oil with CBD or other cannabinoid (e.g., by applying heat and/or cooling the mixture). The carrier oil can be or include, for example, a medium-chain triglyceride (MCT) oil, palm oil, coconut oil, hemp seed oil, an omega-3 fatty acid (e.g., flaxseed or fish oil), a long-chain triglyceride (LCT), glycerin, or any combination thereof. MCT oil can be odorless, colorless, tasteless, and non-allergenic, and these properties can be conferred to the final product. Compared to other carrier oils, MCT oil can be more stable, not prone to oxidative rancidity or off-notes, and/or more readily absorbed by the body. In some examples, the carrier oil can include one or more antioxidants, such as tocopherols (e.g., at about 0.1-0.4 wt. %), diterpenes (e.g., carnosic acid), fatty acid esters of flavonoids (e.g., palmitoylated catechins), or alkyl esters of phenolic acids (e.g., pentyl gallate). The type of carrier oil or combination of carrier oils selected and used can influence emulsion stability and/or absorption into the body or blood stream. In some instances, the carrier oil may provide nutritional or health benefits. The CBD or other cannabinoid can be or include an isolate (e.g., a crystalline, solid form of pure CBD) and/or an oil (e.g., CBD oil, distillate, concentrate, or other cannabinoid oil). The CBD isolate or other cannabinoid isolate can be colorless and generally odor-free. In general, it can be desirable to use ingredients that do not impart color, odor, or taste into the DCE or final products produced from the DCE. The carrier oil and CBD (or other cannabinoid) can be combined at a ratio of about 1:1, by weight, though other proportions can be used (e.g., from about 1:0.1 to about 1:10).

At step 106, the water phase and the oil phase are mixed in a high-speed mixer to produce a coarse emulsion 107 (e.g., an O/W emulsion having relatively large particles and/or large particle size variations) by causing a shearing effect. Heating and or cooling may be applied at this step. The high-speed mixer can be, for example, a SILVERSON shear mixer (or other suitable device), and/or can be operated at one or more speeds (e.g., about 10,000 to about 20,000 RPM) for about 5 to 10 minutes. In some examples, the water phase can be introduced to the mixer first, and the oil phase can be added (e.g., dropwise) while the mixer is operating. The coarse emulsion 107 can have oil particle sizes ranging from about 2000 nm to 200,000 nm. The oil phase and water phase can be present in the coarse emulsion 107 at a ratio of about 1:40 (e.g., about 2.4% oil), by weight, though other proportions can be used (e.g., from about 1:4 to about 1:400).

At step 108, the coarse emulsion 107 can be converted into a fine emulsion 109 (e.g., an O/W emulsion having relatively small particle sizes and/or a small particle size distribution) using an ultrasonic homogenizer, a membrane, a microfluidizer, emulsification, or a high-pressure homogenization device (or other suitable device), such as a STANSTED high-pressure homogenizer (available from HOMOGENISING SYSTEMS LTD., Essex UK). Particle size reduction is typically achieved through a shearing mechanism. The high-pressure homogenization device can include, for example, a heat exchanger, a piston, a nozzle, a microchannel, an interaction zone, and/or a mixing chamber. The oil and water phases can be subjected to pressures ranging from about 10,000 psi to about 60,000 psi and/or temperatures ranging from about 25° C. to about 65° C. in the high-pressure homogenization device. For some formulations, higher temperatures (e.g., from about 45° C. to about 65° C.) can aid in emulsion particle formation and size reduction. The oil and water phases can be passed through the high-pressure homogenization device multiple times (e.g., about 10), until an emulsion of suitable quality or having desired particle sizes is achieved. The fine emulsion 109 can have oil particle sizes ranging from about 10 nm to about 400 nm, or from about 20 nm to about 200 nm, for example, with an average size of about 100 nm and/or a percent polydispersity index less than about 20. The small particles and/or small particle size distribution of the fine emulsion 109 can promote emulsion stability and facilitate absorption into the body or blood stream.

In some examples, the coarse emulsion 107 and/or the fine emulsion 109 can include one or more stabilizers, such as polysaccharides (e.g., gum arabic). The stabilizers may not be surface-active but can impart long-term stability to emulsions by resisting interfacial interactions. For example, the stabilizer can facilitate emulsion formation and/or promote emulsion stability by preventing or minimizing phase separation. A variety of natural or synthetic stabilizers and/or emulsifiers can be used alone or in combination. Additionally or alternatively, one or more natural or synthetic preservatives can be included in the emulsions, such as, for example, NAGARDO (glycolipids), ascorbic acid, ascorbyl palmitate, sodium benzoate, BHT, TBHQ, and/or sorbate. The preservatives can improve chemical, physical, and/or microbiological stability.

At step 110, the fine emulsion 109 can be mixed with a filler or bulking agent, which can act as a carrier and/or can assist with drying steps described herein. The bulking agent can be or include, for example, maltodextrin, a maltodextrin surrogate, starch (e.g., simple and complex sugars), protein (e.g., animal, whey, vegetable, pea, potato, hemp, etc.), fiber (e.g., chitin, cellulose, hemicellulose, inulin, fructans, gums, polyuronides, raffinose, polydextrose, etc.), and/or their modified forms and derivatives. For example, maltodextrin can be dissolved in the fine emulsion 109 (e.g., in the water phase) in small batches and/or by waiting for a period of time (e.g., about 12 hours). The filler and water phase can be present in the fine emulsion 109 at a ratio of about 1:10 (e.g., the water or matrix phase can include about 9% filler), by weight, though other proportions can be used (e.g., from about 1:5 to about 1:20). Adding the bulking agent after the fine emulsion 109 has been formed can allow particles of a desired size to be formed at step 108 and then coated or further encapsulated with the bulking agent at step 110. Otherwise, if the bulking agent is added before step 108, the bulking agent can interfere with the emulsification process and/or can make it difficult or impossible to achieve the desired particle sizes.

At step 112, the mixture can be pasteurized (e.g., with UV light, heat, or gamma irradiation), if desired. In some examples, flash or high-temperature short-time (HTST) pasteurization can be preferred. Other suitable pasteurization techniques may include retort, high pressure pasteurization, use of a plate and frame heat exchanger, or refractance window drying.

Finally, at step 114, the mixture can be dried in an RWD process utilizing a refractance window dryer. In certain examples, the dryer can include a drying chamber containing a heated medium (e.g., a hot water bath), a moving belt (e.g., a PET belt) disposed over or floating on the heated medium, an air supply (e.g., an air supply manifold for providing dry air), and an air exhaust (e.g., an air exhaust manifold for removing moist air). The RWD process can involve spreading the mixture on the moving belt (e.g., using a coating or spreading device) and allowing the heated medium to heat the belt and the mixture. Water evaporates from the mixture as the belt and mixture are heated and travel through the dryer. The dried mixture is removed from the belt at the dryer exit (e.g., using a scraper or blade). RWD technology is described in U.S. Pat. No. 11,221,179, issued on Jan. 11, 2022, the entire disclosure of which is incorporated by reference. In various examples, the RWD process converts the wet mixture into DCE 115, which can be in the form of glassy dried flakes. Alternatively or additionally, one or more other types of dryers or drying techniques can be used, such as, for example, spray drying, drum drying, or freeze drying.

Referring to Table 1, an exemplary DCE 115 produced according to the method 100 can include a cannabinoid, a bulking agent, water, a carrier oil, and an emulsifier. Each ingredient listed in the table may be present within the range of “low” and “high” values, with “typical” describing the average value observed.

TABLE 1
Composition of DCE 115.

| Ingredient | Low | Typical | High |
|---|---|---|---|
| Cannabinoid (wt %) | 1 | 10 | 40 |
| Bulking agent (wt %) | 10 | 54 | 80 |
| Water (wt %) | 0 | 3 | 6 |
| Carrier oil (wt %) | 1 | 22 | 40 |
| Emulsifier (wt %) | 1 | 11 | 20 |

In some embodiments, the DCE described herein (e.g., DCE 115) or a composition comprising the DCE comprises a cannabinoid (or other active) in an amount ranging from about 1% to about 40%, by weight. In an embodiment, a cannabinoid (or other active) may be present in the DCE in an amount greater than, less than, or equal to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, by weight.

In some embodiments, the DCE described herein or a composition comprising the DCE comprises a bulking agent in an amount from about 10% to about 80%, by weight. In an embodiment, a bulking agent may be present in the DCE in an amount greater than, less than, or equal to about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, or about 80%, by weight.

In other embodiments, the DCE described herein or a composition comprising the DCE comprises water in an amount ranging from about 0% to about 6%, by weight. In an embodiment, water may be present in the DCE in an amount greater than, less than, or equal to about 0.005%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, or about 6%, by weight.

In still other embodiments, the DCE described herein or a composition comprising the DCE comprises a carrier oil in an amount ranging from about 1% to about 40%, by weight. A carrier oil may be present in the DCE in an amount greater than, less than, or equal to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40%, by weight.

In some embodiments, the DCE described herein or a composition comprising the DCE comprises an emulsifier in an amount ranging from about 1% to about 20%, by weight. An emulsifier may be present in the DCE or the composition comprising the DCE in an amount greater than, less than, or equal to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%, by weight.

In some embodiments, the DCE described herein or a composition comprising the DCE may include emulsion particles encapsulated in a matrix phase, which can include water and/or a filler or bulking agent (e.g., maltodextrin). The DCE described herein may have a moisture content of less than about 6%, less than about 5%, less than about 4%, less than about 3%, or less than about 2%, by weight. In some aspects, the moisture content may be about 3%, by weight. Water activity for the DCE described herein may range from about 0.1 to about 0.7, or from about 0.3 to about 0.5, or about 0.4. The DCE described herein may be dry to the touch. Actives (e.g., CBD or other cannabinoid) may be present in the DCE described herein in an amount that is less than or equal to about 40%, less than or equal to about 30%, less than or equal to about 20%, less than or equal to about 15%, less than or equal to about 10%, or less than or equal to about 5%, by weight. In some aspects, actives may be present in the DCE in an amount from about 8% to about 12%, by weight.

In some embodiments, the DCE described herein may be comprised of or may comprise flakes or particles of an amorphous solid and/or glassy solid. The flakes or particles can be glossy and/or clear, white-clear, or tinged light yellow or light brown. Flakes may be colored by addition of water soluble or fat soluble pigments (e.g., at step 102 or 104). In certain implementations, use of the RWD process can result in the DCE having a glassy, crystalline appearance, rather than a dull powder appearance (e.g., obtained using spray drying or other drying techniques). Additionally or alternatively, the DCE may have particle sizes that are larger than particles present in a powder (e.g., produced by spray drying). The DCE described herein may have minimum, maximum, or average particle sizes ranging from, for example, about 100 μm to about 1000 μm or more, or can have particles sized to achieve 90% passage through a 40 mesh (400 μm). The larger particle sizes (e.g., coupled with hydrophilic characteristics of the matrix phase) can make the flakes easier to pour and/or disperse in water, compared to powders. For example, when added to an aqueous liquid (e.g., water), the particles may be easily contacted and/or wetted by the aqueous liquid, which can cause the particles to sink and dissolve. By comparison, a powder may not be easily contacted or wetted by the aqueous liquid, such that the powder may not sink and/or can be difficult to mix with the aqueous liquid. Powders can present dust, inhalation, and explosion hazards. In certain embodiments, the flakes or particles of the DCE described herein can be milled to achieve any desired particle sizes or range of particle sizes, including ranges associated with powders (e.g., 20 μm to 80 μm). Additionally or alternatively, the DCE described herein can be blended with other ingredients (e.g., a different dry cannabinoid emulsion and/or a liquid and/or dry colorant) to modify the composition or properties of the composition. In some examples, colorants may be introduced at other stages of the method 100. For example, depending on a colorant's solubility in water or oil, a colorant can be added to the water phase (e.g., water and emulsifier) or the oil phase (e.g., carrier oil and cannabinoid) described above for steps 102, 104, and 106). In other examples, a colorant may be added to the filler or bulking agent (e.g., at step 110).

Advantageously, compared to prior compositions, the water-insoluble cannabinoid in the DCE described herein may be more water compatible, which can improve bioavailability. The DCE can be reconstituted with aqueous media to enable oral, dermal, ocular, and other routes of delivery. In some embodiments, the DCE described herein can be reconstituted into a beverage, added to foods, or incorporated into cosmetics, topicals, lotions, etc., for human and/or animal use. In certain implementations, the DCE can be formed into a pill, capsule, or tablet and consumed orally. The DCE may be formed into a suppository and may be consumed anally or vaginally.

In various implementations, a system for producing the DCE described herein can include any of the materials and equipment described above for the method 100. For example, the system can include one or more of the following, in any combination: a source of water, a source of emulsifier, and a mixer and/or container for performing step 102; a source of carrier oil, a source of a cannabinoid (e.g., CBD), and a mixer and/or container for performing step 104; a high-speed mixer for performing step 106; a homogenization device for performing step 108; a source of filler or bulking agent and a mixer for performing step 110; a pasteurization device for performing step 112; a refractance window dryer for performing step 114; and a mill, a mixer, or a blender to adjust particle sizes and/or blend the DCE with other ingredients.

### Cannabinoid-Cyclodextrin Complexes

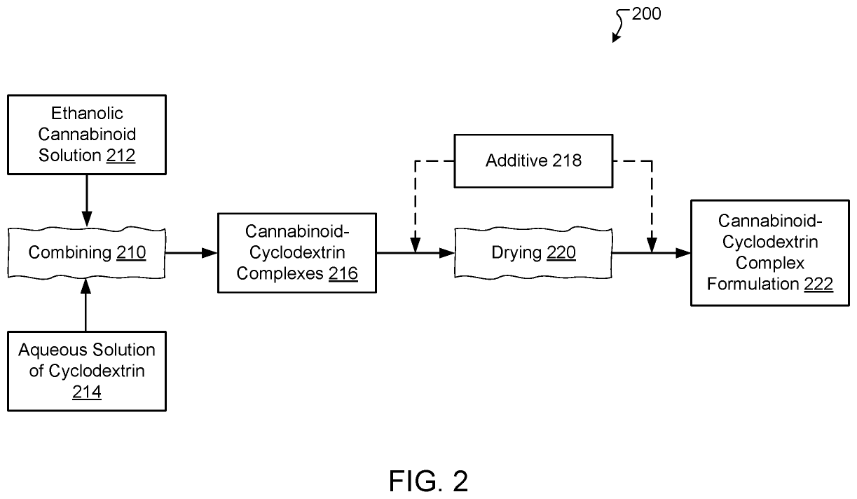

Referring to FIG. 2, the present disclosure provides a method 200 for producing a cannabinoid-cyclodextrin complex (e.g., DC-GCD). The method 200 may include combining (step 210, e.g., by dropwise addition) an ethanolic solution of CBD 212 (or other cannabinoid) with an aqueous solution of cyclodextrin 214. As the ethanolic solution 212 is added to the aqueous solution 214, ring-shaped cyclodextrin molecules can encapsulate CBD molecules, and the resulting CBD-cyclodextrin complexes 216 can precipitate out of solution. The ethanolic solution can include, for example, from about 10 mg to about 1000 mg of CBD (or other cannabinoid) per 5 mL of ethanol, or about 100 mg of CBD per 5 mL of ethanol (or about 20 mg of CBD per mL of ethanol). The ethanol in this example can be a mixture of ethanol and water having about 90% to 100% ethanol by volume. In other embodiments, the ethanol in the ethanolic solution can be replaced in whole or in part with a different solvent, such as, for example, methanol, propanol, isopropanol, acetone, propylene glycol, dimethyl sulfoxide (DMSO), or any combination thereof. The aqueous solution of cyclodextrin can include, for example, from about 350 mg to about 1400 mg of cyclodextrin per 40 mL of water, or about 700 mg of cyclodextrin per 40 mL of water (or about 17.5 mg of cyclodextrin per mL of water). The cyclodextrin may be gamma cyclodextrin, which can function like a precision encapsulant for a CBD molecule or other active.

In some embodiments, the gamma cyclodextrin can be combined with or replaced by alpha cyclodextrin, beta cyclodextrin, and/or delta cyclodextrin, and/or alpha, beta, gamma, or delta forms of hydroxypropyl-cyclodextrins, methylated-cyclodextrins, or acetylated-cyclodextrins. Alternatively or additionally, the cannabinoid complexes can be formed under other host-guest chemistry scenarios. For example, the cannabinoid complexes can be formed with calixarenes, dendrimers, cyclic peptides, or cyclic condensed tannins, rather than cyclodextrin. Cyclic peptides can be more biocompatible (e.g., compared to cyclic condensed tannins) and can have nutritional value and certain biological benefits. In some examples, cannabinoids complexed with hydroxypropyl-cyclodextrins may enhance water solubility or may be more water soluble, compared to other cannabinoid complexes.

Additionally or alternatively, the DC-GCD complexes (or other cannabinoid complexes) can be formulated to include one or more other ingredients or additives (step 218), such as encapsulated amylase enzymes, to ensure a quicker breakdown in vivo. The cannabinoid complex formulation may include excipients that improve stability (e.g., antioxidants) and/or bulking agents to increase volume (e.g., maltodextrin, cellulose, sugars, sugar alcohols, etc.).

Alternatively or additionally, the cannabinoid complex formulation can include other enzymes (e.g., instead of or in addition to the amylase enzyme) such as, for example, an amyloglucosidase enzyme, an amylolytic enzyme, one or more commercial enzyme preparations, a probiotic (e.g., Lactobacillus spp.) that includes an enzyme, or any combination thereof. In some instances, the cannabinoid complex formulation can have an enzyme activity level from about 1,000 U to about 1,000,000 U.

After the DC-GCD complexes (or other cannabinoid complexes) have been formed, the DC-GCD complexes can be removed from the aqueous solution (e.g., as a precipitate) and dried. In certain implementations, for example, the cannabinoid complexes can be separated from the aqueous solution using a filter, a centrifuge, a settling device, other separation device, or any combination thereof. Additionally or alternatively, the cannabinoid complexes can be dried (step 220) in a refractance window dryer or other suitable dryer (e.g., a spray dryer, a drum dryer, or a freeze dryer). The cannabinoid complexes can be mixed (e.g., in a mill, a blender, or a V-type mixer) with an enzyme (e.g., an amylase enzyme), an excipient, a bulking agent, and/or other additive, before or after being dried, to obtain the cannabinoid complex formulation 222 described herein.

Referring to Table 2, an exemplary cannabinoid complex formulation (e.g., the cannabinoid complex formulation 222) produced according to the method 200 can include one or more cannabinoid complexes, enzymes, excipients, a bulking agent, and water. Each ingredient listed may be present within the range of “low” and “high” values, with “typical” describing the average value observed.

TABLE 2
Exemplary cannabinoid complex formulation.

| Ingredient | Low | Typical | High |
|---|---|---|---|
| Cannabinoid complex (wt %) | 5 | 15 | 20 |
| Enzymes (wt %) | 0 | 7 | 10 |
| Excipients (wt %) | 0 | 35 | 50 |
| Bulking agent (wt %) | 0 | 40 | 50 |
| Water (wt %) | 0 | 3 | 6 |

In some embodiments, the cannabinoid complex formulation described herein (e.g., the cannabinoid complex formulation 222) comprises one or more cannabinoid complexes (e.g., a DC-GCD complex) in an amount ranging from about 5% to about 20%, by weight. In an embodiment, the one or more cyclodextrin complexes may be present in the cannabinoid complex formulation in an amount greater than, less than, or equal to about 5%, about 6%, about 7%, about 8%, about 9%, about 10%, about 11%, about 12%, about 13%, about 14%, about 15%, about 16%, about 17%, about 18%, about 19%, or about 20%, by weight.

In some embodiments, the cannabinoid complex formulation described herein comprises one or more enzymes (e.g., an amylase enzyme) in an amount ranging from about 0% to about 10%, by weight. In an embodiment, the enzymes may be present in the cannabinoid complex formulation in an amount greater than, less than, or equal to about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10%, by weight.

In some embodiments, the cannabinoid complex formulation described herein comprises one or more excipients in an amount ranging from about 0% to about 50%, by weight. In an embodiment, the excipients may be present the cannabinoid complex formulation in an amount greater than, less than, or equal to about 0.05%, about 0.1%, about 0.5%, about 1%, about 5%, about 10%, about 15%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, by weight.

In some embodiments, the cannabinoid complex formulation described herein comprises a bulking agent, present in an amount ranging from about 0% to about 50%, by weight. In an embodiment, the bulking agent may be present the cannabinoid complex formulation in an amount greater than, less than, or equal to about 0.05%, about 0.1%, about 0.5%, about 1%, about 5%, about 10%, about 15%, about 20%, about 25%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50%, by weight.

In some embodiments, the cannabinoid complex formulation described herein comprises water in an amount ranging from about 0% to about 6%, by weight. In an embodiment, water may be present the cannabinoid complex formulation in an amount greater than, less than, or equal to about 0.005%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, or about 6%, by weight.

The dried cannabinoid complexes and/or cannabinoid complex formulations can be used in a variety of applications, such as, for example, beverages, food products, cosmetics, topicals, lotions, pills, capsules, and/or tablets, for human and/or animal consumption. Advantageously, complexation can attenuate a bitterness that is sometimes associated with cannabinoids.

In some examples, the dried cannabinoid complexes described herein can be water insoluble and, as such, can be absorbed by the body in a manner that differs from how DCEs are absorbed. For example, an oligosaccharide portion of cannabinoid complexes can be degraded in a gut lumen by pancreatic enzymes to liberate free cannabinoids, which can be readily absorbed into a blood stream. Additionally or alternatively, cannabinoid complexes can dissociate in the oral cavity, thereby liberating free cannabinoid for absorption via oral mucosa. The cannabinoid complexes can be used in a variety of oral applications (e.g., lozenges, edible films, hard candies, chewing gum, chewables, etc.).

In various examples, the DC-GCD (or other cannabinoid complex) can be combined with DCE in various proportions (e.g., can be co-dried using RWD) to produce a hybrid cannabinoid ingredient that takes advantage of respective modes of absorption of DCE and DC-GCD. Complementary absorption of actives in the body may facilitate a quicker effect and/or more consistent or repeatable effect. Combining the DCE with DC-GCD can help compensate for differences between people. For example, a group of people can have a more uniform response when a blend of DCE and DC-GCD is used, due to differences in how DCE and DC-GCD can be absorbed by the body.

In certain instances, a hybrid composition can include a mixture of a cannabinoid complex (e.g., DC-GCD) and a DCE (e.g., DCE 115). The cannabinoid complex and the DCE can be present in the hybrid composition in any amounts and/or can be blended with one or more other ingredients (e.g., flavorants, colorants, bulking agents, etc.). In some examples, a ratio of the cannabinoid complex to the DCE in the hybrid composition can range from about 1:99 to about 99:1, or from about 10:90 to about 90:10, or from about 25:75 to about 75:25, or can be about 50:50, by weight.

In various implementations, a system for producing a dried cannabinoid complex or dried cannabinoid complex formulation can include any of the materials and equipment described above for the method 200. For example, the system can include one or more of the following, in any combination: a source of an ethanolic solution of cannabinoid, a source of an aqueous solution of cyclodextrin, and a mixing device and/or container for performing step 210; a source of an additive and a mixing device for performing step 218; and a refractance window dryer for performing step 220.

While much of the description above relates to products that include cannabinoids and, more particularly, CBD, it is understood that other active ingredients (“actives”) can be included in the materials and products described herein, instead of or in addition to CBD or any other cannabinoid. Other possible active ingredients can include, for example, a plant oil, a plant extract, a pigment, a vitamin, an alkaloid (e.g., psilocybin, nicotine, or caffeine), a flavorant, an enzyme, a prebiotic, a probiotic, a postbiotic, a pharmaceutical, a nutraceutical, and/or a cosmetic material.

### Definitions

The term “approximately,”, the phrase “approximately equal to” and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

The indefinite articles “a” and “an,” as used in the specification and in the claims, unless clearly indicated to the contrary, should be understood to mean “at least one.” The phrase “and/or,” as used in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Each numerical value presented herein, for example, in a table, a chart, or a graph, is contemplated to represent a minimum value or a maximum value in a range for a corresponding parameter. Accordingly, when added to the claims, the numerical value provides express support for claiming the range, which may lie above or below the numerical value, in accordance with the teachings herein. Absent inclusion in the claims, each numerical value presented herein is not to be considered limiting in any regard.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. The features and functions of the various embodiments may be arranged in various combinations and permutations, and all are considered to be within the scope of the disclosed invention. Accordingly, the described embodiments are to be considered in all respects as only illustrative and not restrictive. Furthermore, the configurations, materials, and dimensions described herein are intended as illustrative and in no way limiting. Similarly, although physical explanations have been provided for explanatory purposes, there is no intent to be bound by any particular theory or mechanism, or to limit the claims in accordance therewith.

What is claimed is:

1. A method of forming a dry cannabinoid emulsion (DCE), the method comprising:
obtaining a water phase;
obtaining an oil phase comprising a cannabinoid;
forming an oil-in-water emulsion in which the oil phase is suspended as particles in the water phase; and
drying the emulsion in a refractance window dryer to form a dry cannabinoid emulsion (DCE).

2. The method of claim 1, wherein the water phase comprises water and at least one emulsifier, and wherein the at least one emulsifier comprises at least one of a sucrose ester or a saponin.

3. The method of claim 1, wherein the oil phase comprises a carrier oil comprising a medium-chain triglyceride (MCT) oil.

4. The method of claim 1, wherein the cannabinoid comprises a CBD isolate.

5. The method of claim 1, wherein forming the oil-in-water emulsion comprises using a high-pressure homogenization device.

6. The method of claim 1, wherein the particles comprise sizes ranging from about 10 nm to about 400 nm.

7. The method of claim 1, further comprising mixing the oil-in-water emulsion with a bulking agent.

8. A dry cannabinoid emulsion (DCE) composition comprising:
a matrix phase comprising a bulking agent and water; and
particles of an oil phase comprising a cannabinoid and dispersed within the matrix phase, wherein the particles range in size from about 10 nm to about 400 nm.

9. The composition of claim 8, wherein the composition comprises:
the bulking agent in an amount from about 10% to about 80%, by weight;
the water in an amount from about 0% to about 6%, by weight;
the cannabinoid in an amount from about 1% to about 40%, by weight;
a carrier oil in an amount from about 1% to about 40%, by weight; and
an emulsifier in an amount from about 1% to about 20%, by weight.

10. The composition of claim 8, further comprising an emulsifier comprising at least one of a sucrose ester or a saponin.

11. The composition of claim 8, wherein the oil phase comprises a carrier oil comprising a medium-chain triglyceride (MCT) oil.

12. The composition of claim 8, wherein the cannabinoid comprises a CBD isolate.

13. The composition of claim 8, further comprising a dried cannabinoid complex.

14. A method of forming a dried cannabinoid-cyclodextrin complex, the method comprising:
obtaining an aqueous solution comprising cyclodextrin;
adding a mixture of ethanol and a cannabinoid to the aqueous solution to form a cannabinoid-cyclodextrin complex; and
drying the cannabinoid-cyclodextrin complex in a refractance window dryer to form a dried cannabinoid-cyclodextrin complex.

15. The method of claim 14, wherein the cyclodextrin comprises at least one of gamma cyclodextrin, alpha cyclodextrin, beta cyclodextrin, or hydroxypropyl-cyclodextrin.

16. The method of claim 14, further comprising mixing the cannabinoid-cyclodextrin complex with at least one of an amylase enzyme, an excipient, or a bulking agent.

17. A composition comprising:
a dried cannabinoid-cyclodextrin complex; and
at least one of an amylase enzyme, an excipient, or a bulking agent mixed with the dried cannabinoid-cyclodextrin complex.

18. The composition of claim 17, wherein the composition comprises:
the dried cannabinoid-cyclodextrin complex in an amount from about 5% to about 20%, by weight;
the amylase enzyme in an amount from about 0% to about 10%, by weight;
the excipient in an amount from about 0% to about 50%, by weight; and
the bulking agent in an amount from about 0% to about 50%, by weight.

19. The composition of claim 17, wherein the dried cannabinoid-cyclodextrin complex comprises at least one of alpha cyclodextrin, beta cyclodextrin, gamma cyclodextrin, delta cyclodextrin, alpha hydroxypropyl-cyclodextrin, beta hydroxypropyl-cyclodextrin, gamma hydroxypropyl-cyclodextrin, delta hydroxypropyl-cyclodextrin, a methylated-cyclodextrin, or an acetylated-cyclodextrin.

20. The composition of claim 17, further comprising a dry cannabinoid emulsion (DCE).
